(19) United States
(12) Patent Application Publication
(10) Pub. No.: US 2024/0264074 A1
(43) Pub. Date: Aug. 8, 2024

(54) RAPID MEASUREMENT OF COAL OXIDATION

(71) Applicant: THE UNIVERSITY OF QUEENSLAND, St. Lucia (AU)

(72) Inventors: Yongjun PENG, Queensland (AU); Xumeng CHEN, Queensland (AU)

(21) Appl. No.: 18/562,197

(22) PCT Filed: May 20, 2022
(86) PCT No.: PCT/AU2022/050490

§ 371 Date: Nov. 17, 2023

(30) Foreign Application Priority Data
May 20, 2021 (AU) 2021901508

(51) Int. Cl. G01N 21/33; B01D 11/04; G01N 33/24

(52) U.S. Cl. CPC G01N 21/33; B01D 11/0492; G01N 33/24

(57) ABSTRACT

The present invention relates to a method for determining oxidation of coal, the method comprising: a) mixing a coal sample with an organic solvent and an inorganic solvent, to extract oxidised coal species from the coal sample into a liquid phase, and b) analysing the liquid phase from step (a) to determine a degree of coal oxidation. The inorganic solvent may comprise an inorganic compound such as a pyrophosphate. The present invention also relates to a method for controlling collector used in a coal flotation process, the method comprising determining a degree of oxidation of coal by the preceding method and controlling a ratio of non-polar collector to polar collector in the coal flotation process in response to the determined degree of oxidation of coal.

### TECHNICAL FIELD

The present invention relates to a method for measuring coal oxidation.

### BACKGROUND ART

Flotation of coal is a commonly used technique to reduce the ash content of coal and to increase the relative amount of combustible material. Flotation of coal in Australia typically uses diesel as a flotation collector. Coal particles having a hydrophobic surface attach to the diesel and float to the top of the flotation vessel, from where they are recovered.

Although non-oxidised surfaces of coal are hydrophobic and float well with diesel being used as the flotation collector, the surface of coal undergoes oxidation once the coal has been unearthed and/or removed from the coal seam. Oxidation of the surface of coal also proceeds during stockpiling and in the subsequent processing during coal production. Surface oxidation of coal is a particularly significant issue at open cut mines.

Although non-oxidised coal has a hydrophobic surface and floats well with oily collectors, oxidised coal has a hydrophilic surface and requires a polar collector to float. Polar collectors have not been widely applied in plants when floating oxidised coals and most coal flotation plants still use diesel as a collector, with poor flotation performance. For best flotation performance, a polar collector should be used together with diesel (or other non-polar collector) and their dosages should be determined by the degree of surface oxidation of the coal. However, there is no known technique available for plant operators to measure the degree of coal oxidation of flotation feed on site.

Initial attempts to develop a method to measure the degree of coal oxidation resulted in the following method:
a) extract coal oxidation species from the coal surface using 1M NaOH solution at 90° C. with agitation,
b) filtering the slurry to get the filtrate, which contains the extracted coal oxidation species,
c) measuring the concentration of coal oxidation species in the filtrate using a laboratory UV/VIS spectrophotometer and determining the degree of surface oxidation from the measured concentration.

Although this process provided accurate measurements of the degree of surface oxidation of the coal, it requires the use of corrosive and hazardous chemicals, the requirement for heating, a long processing time of at least 45 minutes and access to a laboratory. As a result, this process would be difficult to transfer to on-site operation at a coal flotation plant.

It will be clearly understood that, if a prior art publication is referred to herein, this reference does not constitute an admission that the publication forms part of the common general knowledge in the art in Australia or in any other country.

### SUMMARY OF INVENTION

The present invention is directed to a method for measuring coal oxidation, which may at least partially overcome at least one of the abovementioned disadvantages or provide the consumer with a useful or commercial choice.

In a first aspect, the present invention provides a method for determining oxidation of coal, the method comprising:
a) mixing a coal sample with an organic solvent and an inorganic solvent, to extract oxidised coal species from the coal sample into a liquid phase, and
b) analysing the liquid phase from step (a) to determine a degree of coal oxidation.

In one embodiment, the inorganic solvent reacts with oxidised species on the surface of the coal. In one embodiment, the inorganic solvent reacts with oxidised species on the surface of the coal and the extracted species dissolve in water. In one embodiment, the inorganic solvent comprises a complex-forming solution. In one embodiment, the inorganic solvent dissolves coal oxidation species through ion exchange.

In one embodiment, the extracted species dissolve in the organic solvent.

In one embodiment, the inorganic solvent comprises an aqueous solution of an inorganic compound.

Without wishing to be bound by theory, it is postulated that the oxidised species on the surface of the coal react with the inorganic compound and the extracted species dissolve in the mixture of water and organic solvent. It is also postulated that the organic solvent increases the extraction efficiency and solubility of oxidised species and also increases the solubility of oxidised species in water.

In one embodiment, the organic solvent selectively dissolves the oxidised organic species on the surface of the coal, and then the inorganic compound in the inorganic solvent selectively dissolves the oxidised species in the water.

In one embodiment, the inorganic solvent and the organic solvent are miscible.

On the surface of the coal, there are many different organic species, including oxidised species and un-oxidised species. The present invention advantageously selectively dissolves the oxidised species into the liquid, which will allow the liquid to be analysed to determine the amount or concentration of the oxidised species in the liquid. Inorganic solvent can selectively dissolve and extract oxidised species, however, it has a low efficiency. Organic solvent has higher extraction efficiency, however, organic solvent is not selective and can dissolve both oxidised species and un-oxidised species. The present inventors have found that combining the inorganic compound together with organic solvent achieves a good extraction efficiency while maintaining the selectivity to extract the oxidised species into the liquid.

In one embodiment, the coal sample is mixed with a mixture comprising the organic solvent and the inorganic solvent, suitably with the inorganic solvent being in the form of an aqueous solution of the inorganic compound. In this procedure, the role of the organic solvent is to improve the extraction efficiency and solubility of oxidised species.

In one embodiment, the coal sample is first mixed with the organic solvent to extract all coal surface species into solution and then add an aqueous solution of the inorganic solvent to selectively dissolve the oxidised species into the liquid. This procedure is good for measuring dry coal samples.

In one embodiment, step (a) is conducted without requiring external heating. In one embodiment, step (a) is conducted at ambient temperature or at room temperature (such as 20 to 40° C. or 20 to 30° C.). In one embodiment, step (a) is conducted at ambient pressure, or about one atmosphere (101.325 kPa).

In one embodiment, a contact time of less than 10 minutes between the coal sample and the liquid phase may be used, or less than 5 minutes, or less than 4 minutes, or less than 3 minutes, or less than 2 minutes, or about 1 minute. Experimental work conducted by the inventors has shown that a contact time of about 1 minute in step (a) is sufficiently long to obtain good measurement results.

In one embodiment, step (a) is conducted with shaking or agitation.

In one embodiment, the coal sample comprises a coal slurry. The coal slurry may comprise a coal slurry that is being fed to a coal flotation plant. The typical range of coal to water ratios in the coal slurry is from 2 wt % to 15 wt %. In one embodiment, the coal to water ratio in the coal slurry is from 2 wt % to 10 wt %, or about 5 wt %.

In one embodiment, the coal sample is dry coal particles, for example, from the coal mine.

In one embodiment, the inorganic solvent comprises K₄P₂O₇ (potassium pyrophosphate) or Na₄P₂O₇ (sodium pyrophosphate), Na₂CO₃, K₂CO₃, Na₂B₄O₇ or K₂B₄O₇; or mixtures thereof, or solutions thereof, or aqueous solutions thereof. In one embodiment, the inorganic solvent comprises K₄P₂O₇ (potassium pyrophosphate) or Na₄P₂O₇ (sodium pyrophosphate), or mixtures thereof, or aqueous solutions thereof. In one embodiment, the inorganic solvent comprises carbonate, pyrophosphate, or tetraborate salts, or mixtures thereof, or solutions thereof, or aqueous solutions thereof. Other inorganic solvents may be used. The inorganic solvent may be an aqueous solution.

The inorganic solvent may comprise an inorganic compound selected from K₄P₂O₇, Na₄P₂O₇, Na₂CO₃, K₂CO₃, Na₂B₄O₇ and K₂B₄O₇, or mixtures thereof. The inorganic solvent may comprise an inorganic compound selected from K₄P₂O₇, or Na₄P₂O₇, or mixtures thereof. The inorganic solvent may comprise an inorganic compound comprising a carbonate, a pyrophosphate, or a tetraborate, or mixtures thereof.

The inorganic solvent may comprise an inorganic compound at any suitable concentration. In one embodiment, the inorganic solvent comprises from 0.1 M to 5 M inorganic compound, or from 0.1 M to 2 M inorganic compound, or from 0.1 to 1 M inorganic compound, or about 0.5M inorganic compound, or about 0.25M inorganic compound.

In one embodiment, the organic solvent comprises an organic solvent that has good solubility of extracted species of oxidised coal, is miscible with water and does not affect the measurement technique used to determine the concentration of extracted oxidised coal species in solution. In one embodiment, the organic compound does not affect a UV/VIS spectrophotometry measurement at the wavelength used for that measurement.

In one embodiment, the organic solvent is selected from one or more of ethanol, methanol, 1-propanol, 2-propanol, dioxane, dimethyl sulfoxide, tetrahydrofuran, and dimethylformamide. In one embodiment, the organic solvent is ethanol.

In one embodiment, step (a) produces a mixture in which the coal to liquid ratio is from 0.5 wt % to 10 wt %, or about 1 wt % to 5 wt %, or about 1 wt % to about 4 wt %.

In one embodiment, step (a) produces a mixture in which the percentage by volume of the organic solvent is from about 3 vol % to about 40 vol %, or from about 3 vol % to about 30 vol %, or from about 5 vol % to about 30 vol %, or from about 8 vol % to about 30 vol %.

In one embodiment, step (a) produces a mixture in which the concentration of inorganic compound is from 0.005M to 1M, or from 0.005M to 0.5M, or from 0.01M to 0.2M, or from 0.01M to 0.1M.

Without wishing to be bound by theory, in some embodiments the present inventors have postulated that the oxidised coal species on the surface of the coal react with the inorganic compound in the inorganic solvent and the extracted species dissolve in water, while the organic solvent increases the extraction efficiency of the inorganic solvent and increases the solubility of oxidised species in water. In some embodiments the present inventors have postulated that the organic species on the surface of the coal dissolve in the organic solvent, and then the inorganic compound in the inorganic solvent selectively react with only oxidised coal species and dissolve them in water. It is further postulated that adding water causes the unoxidised species to become essentially insoluble in the mixture of organic solvent and water whilst the oxidised species react with the inorganic compound in solution and thus the oxidised species can remain in solution.

Without wishing to be bound by theory, the present inventors have postulated that aqueous solutions of sodium hydroxide, potassium hydroxide and the like make hydroxycarboxylic acids on oxidised coal water soluble by ionising the acids. Furthermore, the present inventors postulate that inorganic solvents comprising inorganic compounds such as Na₄P₂O₇, K₄P₂O₇, and sodium tetraborate may form complexes with polyvalent metal ions in oxidised coal and/or break electrostatic bonds in oxidised coal. For example, oxidised coal may comprise humic substances and polyvalent metal ions (such as Ca²⁺ and Mg²⁺) may link such humic substances to inorganic colloids. The inventors believe that inorganic compounds such as pyrophosphate may form complexes with such humic substances and assist in replacing the polyvalent ions with monovalent cations (such as sodium), which thereby increases aqueous solubility of the oxidised coal. The inventors further believe that some organic solvents may assist in breaking the intermolecular bonds of the oxidation species on oxidised coal.

In preferred embodiments, step (a) can be conducted at room temperature without requiring use of hazardous chemicals and utilising a contact time of around 1 minute. This makes step (a) suitable for use on a flotation plant site.

In one embodiment, step (b) involves analysing the liquid phase from step (a) to determine the concentration of coal oxidation extracted species in the liquid phase. The degree of oxidation of the coal sample can then be determined from the results of this analysis.

In one embodiment, the liquid phase from step (a) is separated from the coal sample prior to step (b). In one embodiment, the mixture of coal sample and liquid in step (a) is filtered to remove coal particles from the liquid phase. Other solid/liquid separation techniques may also be used.

In one embodiment, step (b) uses UV/VIS spectrophotometry to determine the concentration of extracted species in the liquid phase. In one embodiment, a single wavelength UV/VIS spectrophotometer is used in step (b). In one embodiment, a portable, single wavelength UV/VIS spectrophotometer is used in step (b). In one embodiment, the wavelength of the UV/VIS spectrophotometer is from about 250 nm to about 270 nm; especially at about 254 nm or about 270 nm. The inventors have advantageously found that any wavelength from about 250 nm to about 270 nm is suitable.

In one embodiment, the UV absorbance of the liquid phase is measured in step (b).

In one embodiment, the analysis used in step (b) provides a value of the degree of surface oxidation of the coal. In another embodiment, the analysis used in step (b) is used to correlate a degree of surface oxidation of the coal. Another embodiment, the analysis used in step (b) is used to determine a degree of surface oxidation of the coal.

The first aspect of the present invention may provide a rapid method for determining a degree of surface oxidation of coal. The method can be implemented on-site at a coal flotation plant. The method can provide quasi-real-time analysis of the degree of surface oxidation of coal being fed to a coal flotation plant.

In a second aspect, the present invention provides a method for controlling collector used in a coal flotation process, the method comprising determining oxidation of coal in accordance with the first aspect of the present invention and controlling a ratio of non-polar collector to polar collector in the coal flotation process in response to the determined oxidation of coal.

In one embodiment of the second aspect, the present invention provides a method for controlling collector used in a coal flotation process, the method comprising determining a degree of surface oxidation of coal in accordance with the first aspect of the present invention and controlling a ratio of non-polar collector to polar collector in the coal flotation process in response to the determined degree of surface oxidation of the coal.

In one embodiment of the second aspect of the present invention, the method comprises regularly or frequently determining the degree of surface oxidation of the coal in accordance with the first aspect of the present invention and, if a subsequent determination shows more surface oxidation of the coal than a previous determination, decreasing the ratio of non-polar collector to polar collector, and, if a subsequent determination shows less surface oxidation of the coal than a previous determination, increasing the ratio of non-polar collector to polar collector. In other words, if the degree of surface oxidation is determined to have increased, the amount of polar collector is increased relative to the non-polar collector in the flotation step. If the degree of surface oxidation is determined to have decreased, the amount of polar collector is decreased relative to the non-polar collector in the flotation step.

Any known polar collectors and non-polar collectors that are used in coal flotation plants can be used in the present invention. An exemplary non-polar collector includes diesel. Exemplary polar collectors include reagents based on sorbitan esters, fatty acids and fatty acid esters.

The method of the second aspect of the present invention advantageously allows the yield of the coal flotation process to be increased by regularly or frequently determining the degree of oxidation of the coal and adjusting the collector mixture used in the flotation step to increase the amount of coal recovered from the flotation step.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 shows XPS spectra of heavily oxidised coal;

FIG. 2 shows a schematic diagram illustrating diesel or other non-polar collector attaching to unoxidised coal surface and a polar collector attaching to oxidised coal surface;

FIG. 3 shows a graph of degree of oxidised carbon determined by XPS spectroscopy vs spectroscopy oxidation index obtained by analysing coal using the NaOH-based technique described in the background art section of this specification;

FIG. 4 shows a graph of UV absorbance determined from experimental work in accordance with an embodiment of the present invention plotted against UV absorbance determined using the NaOH-based technique described in the background art section of this specification;

FIG. 5 shows a graph of UV Spectroscopy readings using a portable UV spectrophotometer vs UV spectroscopy readings using a laboratory UV spectrophotometer;

FIG. 6 shows a graph of combustible recovery vs flotation time using a heavily oxidised coal feed and varying ratios of diesel collector and polar collector;

FIG. 7 shows a graph of combustible recovery vs flotation time using a medium oxidised coal feed and varying ratios of diesel collector and polar collector;

FIG. 8 shows a graph of combustible recovery vs flotation time using a lightly oxidised coal feed and varying ratios of diesel collector and polar collector; and

FIG. 9 shows a graph of UV absorbance of flotation feed in a plant processing old tailings at different times and dates over a one month period.

### DESCRIPTION OF EMBODIMENTS

Preferred embodiments of the present invention were developed with a view to providing a more user-friendly and safe coal oxidation measurement technique that can be used on-site in a plant, such as a coal flotation plant, without requiring laboratory facilities. Preferred embodiments of the present invention require no heating, require only a short reaction time and can use portable apparatus, whilst also requiring minimal operator training.

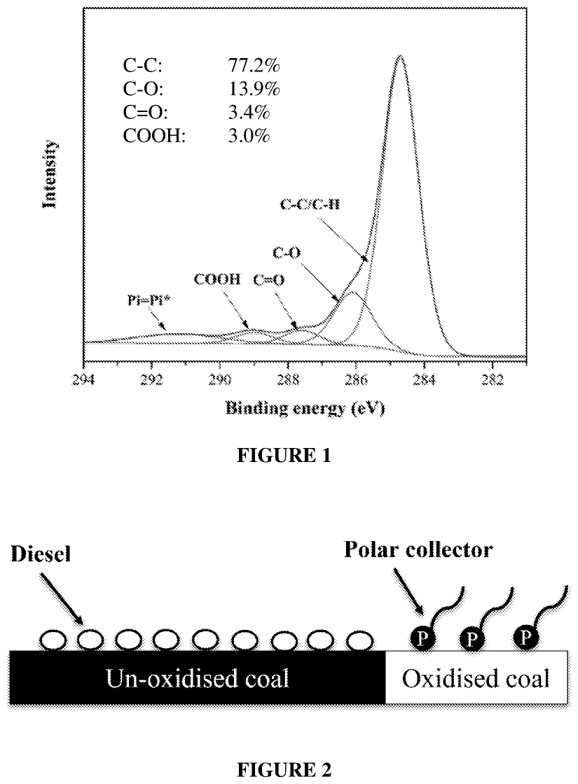

FIG. 1 shows the XPS spectra of heavily oxidised coal. Oxidised coal is widely present in coal mines. Oxidation occurs once coal is in contact with oxygen. It occurs during mining, in stockpiling, during transport and in tailings dams. As can be seen from FIG. 1, a number of oxidised species are present on the surface of oxidised coal.

Oxidised surfaces of coal are generally hydrophilic, whereas non-oxidised surfaces of coal are generally hydrophobic. As a result, different types of flotation collectors are attracted to the surfaces. FIG. 2 shows that a diesel collector (or indeed, other oily or non-polar collectors) is attracted to the non-oxidised surface of coal. In contrast, non-polar collectors are attracted to the oxidised surfaces of the coal. Therefore, strategies to increase the flotation performance of oxidised coals should ideally include use of a mixture of non-polar collectors and polar collectors, with the dosage of non-polar collectors being determined by the degree of surface oxidation. This flotation process would desirably include a rapid method for measuring coal oxidation in order to be able to rapidly respond to changes in the degree of oxidation of the coal feed to the flotation plant.

Preferred embodiments of the present invention were developed with the aim of being able to measure oxidation of coal in time periods of 10 minutes or less.

The first step of the method of preferred embodiments of the present invention involves mixing a coal sample, which will typically be a coal slurry that is being fed to a coal flotation plant, with an inorganic solvent and an organic solvent. The inorganic solvent of the preferred method of the present invention comprises potassium pyrophosphate, K₄P₂O₇. Sodium pyrophosphate may also be used but potassium pyrophosphate is preferred as it has a higher water solubility.

The organic solvent may comprise ethanol. In preferred embodiments of the present invention, the organic solvent is miscible with water and does not interfere with the UV spectroscopy readings that are subsequently taken. DMSO (dimethyl sulfoxide) and several other organic solvents previously named in this specification may also be used in preferred embodiments of the present invention.

In order to measure the UV absorbance of the liquid phase following extraction of coal, a portable UV254 instrument was purchased from Photonic Measurements Ltd in the United Kingdom. This instrument is designed to measure organic carbon in water treatment plants by measuring UV absorbance at 254 nm. Coal oxidation species are mainly aromatic organic carbons which also have strong UV absorbance at 250 nm through to 270 nm, making an instrument that operates at a wavelength of 254 nm suitable for purpose. This instrument is portable, can be directly used in the plant, relatively inexpensive, resistant to dust and water and powered by a battery.

The following procedure was used to measure the degree of coal oxidation:
1) bring the portable UV spectrophotometer and sampling bottles to the coal flotation plant.
2) collect coal slurry sample in the plant and add 25 ml of coal slurry into a 50 ml plastic tube or bottle (other size bottles may also be used).
3) add 2 ml ethanol and 0.8 ml 0.5M K₄P₂O₇ solution into the bottle containing the slurry sample and shake for 1 minute.
4) turn on the UV254 portable spectrophotometer.
5) add the extraction solution having the same ratio of ethanol and K₄P₂O₇ as used in step (3) into the cuvette to use the UV254 portable spectrophotometer to measure the UV absorbance of the blank solution.
6) use a 10 ml syringe to take around 5 ml slurry from the plastic tube or bottle and use a 0.45 μm filter head to filter it, then add the filtrate to the cuvette for UV measurement.
7) measure the UV absorbance of the filtrate.

The above test can be done in the plant and takes around 5 minutes in total. This can be very beneficial for the plant when processing oxidised coals. Plant operators can measure the oxidation degree more frequently and optimise operating conditions based on the degree of coal oxidation. In addition, the extracting agents (ethanol and K₄P₂O₇) are much safer to use compared to 1M NaOH solution. Further, the above method does not require any heating.

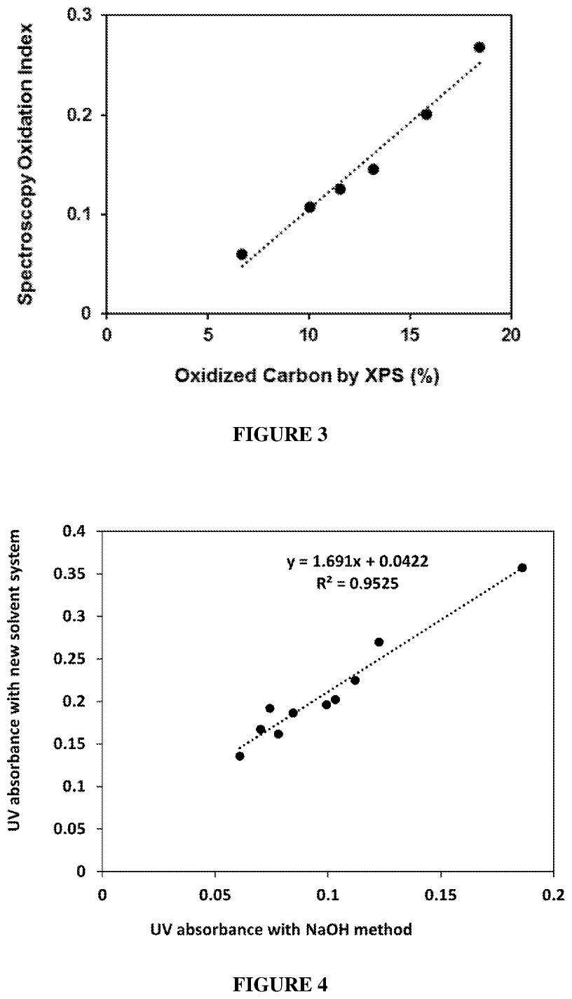

FIG. 3 shows a graph of degree of oxidised carbon determined by XPS spectroscopy vs spectroscopy oxidation index obtained by analysing coal using the NaOH-based technique described in the background art section of this specification. The results shown in FIG. 3 demonstrate that there is a good correlation between the spectroscopic oxidation index obtained using a laboratory UV spectrophotometer following extraction with hot NaOH for 45 minutes and the degree of oxidised carbon determined by XPS. FIG. 4 shows a graph of UV absorbance determined from experimental work in accordance with an embodiment of the present invention plotted against UV absorbance determined using the NaOH-based technique described in the background art section of this specification. As can be seen from FIG. 4, the UV absorbance using the method of preferred embodiments of the present invention provides a good correlation with the UV absorbance measured using the NaOH solvent technique. Therefore, the results of FIGS. 3 and 4 demonstrate that there will be a good correlation between the UV absorbance determined in accordance with preferred embodiments of the present invention and the degree of oxidised carbon in the coal determined by XPS. Further, the correlation between the spectroscopic oxidation index obtained using the NaOH-based method and the degree of oxidised carbon determined by XPS can be used to provide a correlation between the UV absorbance determined in accordance with embodiments of the method of the present invention and the degree of oxidised carbon.

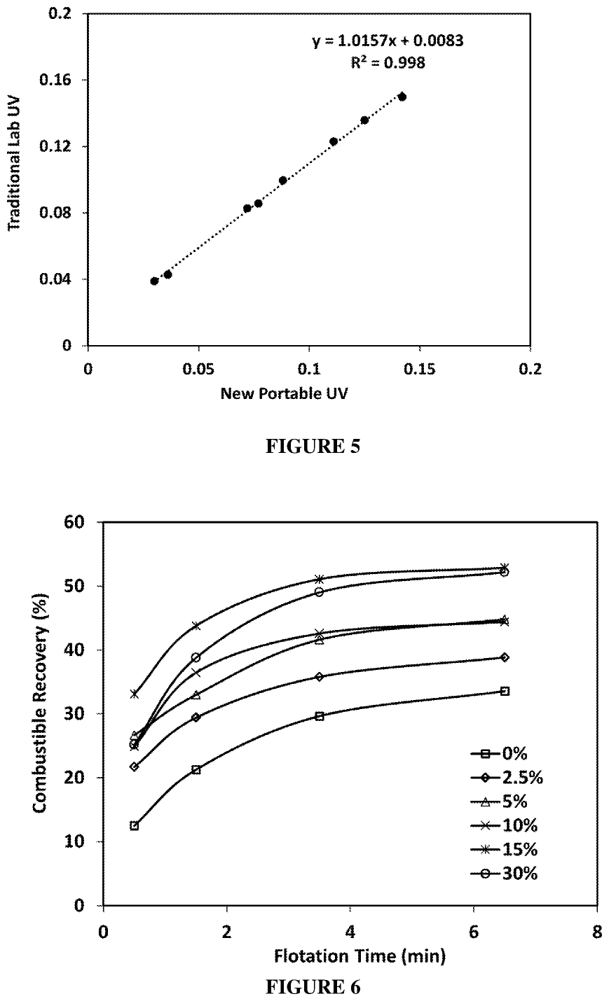

FIG. 5 shows that the portable, inexpensive UV spectrophotometer used in this experimental work gives results that are closely correlated with the laboratory UV spectrophotometer used in previous work. Therefore, the portable, inexpensive UV spectrophotometer used in this experimental work is capable of providing good quality results.

FIG. 6 shows combustible recovery against flotation time for a highly oxidised coal obtained from old tailings reject. The coal sample contains 63% ash in the flotation feed and has a spectroscopy oxidation index of 0.236. The flotation results shown in FIG. 6 use a collector that comprises diesel alone as the collector (the lowest line in the graph) or a mixture of diesel and polar collector, with the amount of polar collector being indicated on the graph. The total collector dosage was fixed at 300 g/t. As can be seen from FIG. 6, adding a polar collector to the diesel increases combustible recovery. The optimum polar collector ratio for the highly oxidised coal was around 15%.

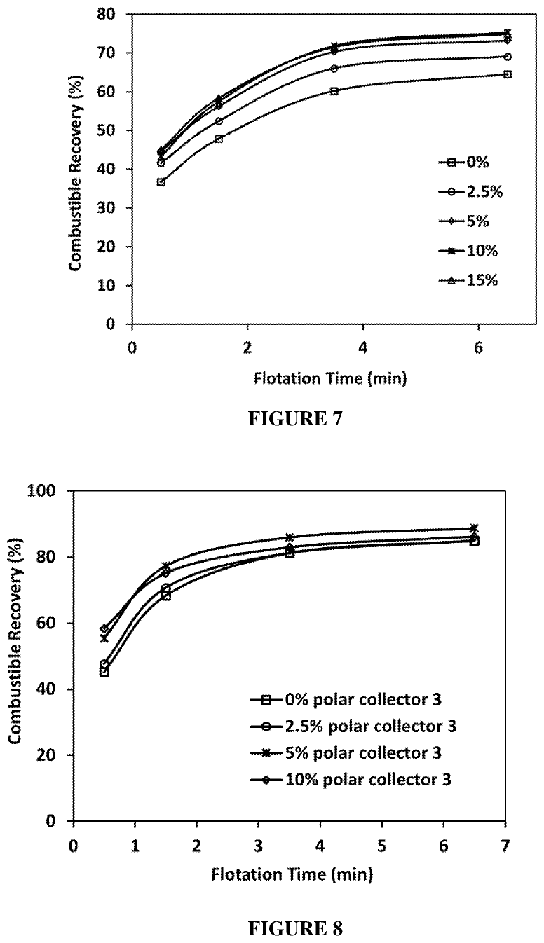

FIG. 7 shows combustible recovery against flotation time for a coal having medium oxidation. The coal sample used in the experimental work shown in FIG. 6 contains 36% ash in the flotation feed and had a spectroscopy oxidation index of 0.190. The flotation results shown in FIG. 7 use a collector that comprises diesel alone as the collector (the lowest line in the graph) or a mixture of diesel and polar collector, with the amount of polar collector being indicated on the graph. The total collector dosage was fixed at 300 g/t. As can be seen from FIG. 7, adding a polar collector to the diesel increases combustible recovery. The optimum polar collector ratio for the medium oxidised coal was 5% to 10%.

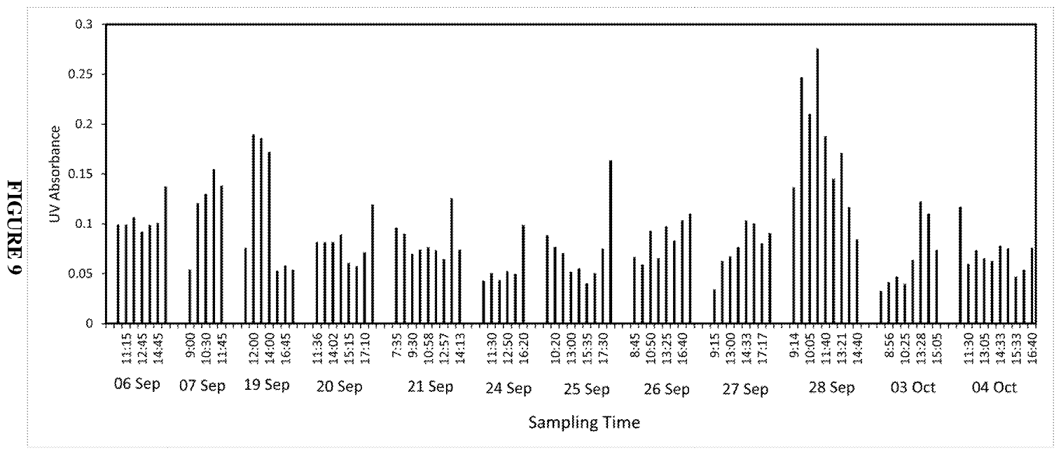

FIG. 8 shows combustible recovery against flotation time for coal having low oxidation. The coal sample used in the experimental work shown in FIG. 8 contains 33% ash in the flotation feed and has a spectroscopy index of 0.122. The flotation results shown in FIG. 8 use a collector that comprises diesel alone as the collector (the lowest line in the graph) or a mixture of diesel and polar collector, with the amount of polar collector being indicated on the graph. The total collector dosage was fixed at 300 g/t. As can be seen from FIG. 8, adding a polar collector to the diesel increases combustible recovery. The optimum polar collector ratio for the highly oxidised coal was around 5%.

The above results demonstrate that the preferred method of the present invention can provide a rapid and easy to use method for determining the degree of coal oxidation. The method can be used at a plant site by a plant operator. The method can provide an accurate determination of the degree of coal oxidation. As the method is rapid, it provides the option of adjusting the ratio of non-polar collector to polar collector used in a flotation plant to optimise flotation recovery.

In another example, the extraction procedure that was tested is as follows:
(1) add 10 ml dimethyl sulfoxide to a tube containing 1 g dry coal, then shake the tube for 30 seconds.
(2) add 25 ml 0.1M K₄P₂O₇ water solution to the tube, shake for another 30 seconds.

In step 1, all organic species on the coal surface are dissolved in dimethyl sulfoxide.

In step 2, because a larger volume of water was added, the un-oxidized organic species became insoluble in the solution, while the oxidized organic species can react with K₄P₂O₇ and therefore remain soluble and the liquid component can be tested for the content/concentration of oxidised species.

In further examples, experiments were conducted with various inorganic chemicals to test their extraction efficiency. This is illustrated in the table below, which used 25 ml aqueous coal slurry (5% solid). The mixture of 4.8 ml 0.5M K₄P₂O₇ (shaking 1 min) provided superior extraction over 100 ml 1M NaOH (heating at 90° C. for 15 min). Even though the mixtures with Na₂CO₃ and Na₂P₂O₇ were not as effective as the NaOH mixture, both of the Na₂CO₃ and Na₂P₂O₇ mixtures were still able to extract the oxidised coal sample, and under conditions that were significantly faster than the NaOH sample (1 min versus 15 min) and at a lower temperature (ambient temperature versus 90° C.). The “Extraction Ranking” in the tables below was determined from a measurement of the absorbance of the solution at 270 nm (after separating solid matter), as the higher the absorbance the more material was extracted into the liquid phase.

| | Extraction |
|---|---|
| Experiment conditions (with Highly Oxidised Coal sample) | Ranking |
| 100 ml 1M NaOH (Heating at 90° C. for 15 min) | 2 |
| 12 ml Ethanol + 4.8 ml 0.5M K₄P₂O₇ (Shaking 1 min) | 1 |
| 12 ml Ethanol + 4.8 ml 0.5M Na₂CO₃ (Shaking 1 min) | 4 |
| 12 ml Ethanol + 4.8 ml 0.5M Na₂P₂O₇ (Shaking 1 min) | 3 |

In other examples, experiments were conducted with different organic chemicals to test their efficiency on extraction. This is illustrated in the table below, which used 25 ml aqueous coal slurry (5% solid). The extractions using methanol, dimethylsulfoxide (DMSO) and tetrahydrofuran (THF) all worked better than the extraction with ethanol. However, all of the organic solvents tested were superior to the NaOH conditions. Ethanol has advantages over THF, DMSO and methanol, as ethanol is less toxic and/or less flammable than these solvents.

| | Extraction |
|---|---|
| Experiment conditions (with Highly Oxidised Coal sample) | Ranking |
| 100 ml 1M NaOH (Heating at 90° C. for 15 min) | 5 |
| 12 ml Ethanol + 4.8 ml 0.5M K₄P₂O₇ (Shaking with 1 min) | 4 |
| 12 ml THF + 4.8 ml 0.5M K₄P₂O₇ (Shaking with 1 min) | 1 |
| 12 ml DMSO + 4.8 ml 0.5M K₄P₂O₇ (Shaking with 1 min) | 2 |
| 12 ml Methanol + 4.8 ml 0.5M K₄P₂O₇ (Shaking with 1 min) | 3 |

In yet more examples, experiments were conducted with different inorganic/organic ratios to test their efficiency on extraction. These results are illustrated in the below tables, which used 25 ml aqueous coal slurry (5% solid). Again, even though the mixtures with 12 ml Ethanol+2.4 ml 0.5M K₄P₂O₇ on an oxidised coal sample and 6 ml Ethanol+4.8 ml 0.5M K₄P₂O₇ on oxidised coal from a tailing dam from a different geographical location were not as effective as the NaOH mixture, both of these solvent mixtures were still able to extract the oxidised coal sample, and under conditions that were significantly faster than the NaOH sample (1 min versus 15 min) and at a lower temperature (ambient temperature versus 90° C.).

| | Extraction |
|---|---|
| | Ranking |
| Experiment conditions (with Highly Oxidised Coal sample) | |
| 100 ml 1M NaOH (Heating at 90° C. for 15 min) | 2 |
| 12 ml Ethanol + 4.8 ml 0.5M K₄P₂O₇ (Shaking with 1 min) | 1 |
| 12 ml Ethanol + 2.4 ml 0.5M K₄P₂O₇ (Shaking with 1 min) | 3 |
| 6 ml Ethanol + 4.8 ml 0.5M K₄P₂O₇ (Shaking with 1 min) | 1 |
| 3 ml Ethanol + 4.8 ml 0.5M K₄P₂O₇ (Shaking with 1 min) | 1 |
| Experiment conditions (with oxidised coal from a tailing | |
| dam from a different geographical location) | |
| 100 ml 1M NaOH (Heating at 90° C. for 15 min) | 2 |
| 12 ml Ethanol + 4.8 ml 0.5M K₄P₂O₇ (Shaking with 1 min) | 1 |
| 12 ml Ethanol + 2.4 ml 0.5M K₄P₂O₇ (Shaking with 1 min) | 1 |
| 6 ml Ethanol + 4.8 ml 0.5M K₄P₂O₇ (Shaking with 1 min) | 3 |
| 12 ml Ethanol + 1.2 ml 0.5M K₄P₂O₇ (Shaking with 1 min) | 1 |

In a further example, experiments were conducted with different concentration of inorganic compounds. The results are illustrated in the below table, which used 25 ml aqueous coal slurry (5% solid). Again, even though the mixture with 12 ml ethanol+4.8 ml 0.1M K₄P₂O₇ was not as effective as the NaOH mixture, this solvent mixture were still able to extract the oxidised coal sample, and under conditions that were significantly faster than the NaOH sample (1 min versus 15 min) and at a lower temperature (ambient temperature versus 90° C.).

| | Extraction |
|---|---|
| Experiment conditions (with Highly Oxidised Coal sample) | Ranking |
| 100 ml 1M NaOH (Heating at 90° C. for 15 min) | 3 |
| 12 ml Ethanol + 4.8 ml 0.5M K₄P₂O₇ (Shaking with 1 min) | 1 |
| 12 ml Ethanol + 4.8 ml 0.25M K₄P₂O₇ (Shaking with 1 min) | 2 |
| 12 ml Ethanol + 4.8 ml 0.1M K₄P₂O₇ (Shaking with 1 min) | 4 |

As discussed above, for best flotation performance, a polar collector should be used together with diesel (or other non-polar collector) and their dosages should be determined by the degree of surface oxidation of the coal. A rapid technique that may allow for on-site determination of coal oxidation therefore may be extremely advantageous. To illustrate this point, FIG. 9 shows the UV absorbance (an indicator of the degree of coal oxidation) of flotation feeds in a coal preparation plant reprocessing old tailing dams. A large number of samples were collected at different times and dates over a one month period. As illustrated in the figure, the degree of oxidation of the flotation feeds varied significantly even in the same day.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

1. A method for determining oxidation of coal, the method comprising:
a) mixing a coal sample with an organic solvent and an inorganic solvent, to extract oxidised coal species from the coal sample into a liquid phase, and
b) analysing the liquid phase from step (a) to determine a degree of coal oxidation.

2. The method of claim 1, wherein the extracted oxidised coal species in step (a) dissolve in the organic solvent.

3. The method of claim 1, wherein the inorganic solvent comprises an aqueous solution of an inorganic compound.

4. The method of claim 3, wherein the inorganic solvent and the organic solvent are miscible.

5. The method of claim 1, wherein step (a) is conducted at ambient temperature.

6. The method of claim 5, wherein in step (a) there is a contact time of less than 5 minutes between the coal sample and the liquid phase.

7. The method of claim 5, wherein step (a) is performed with shaking or agitation.

8. The method of claim 1, wherein the coal sample comprises a coal slurry.

9. The method of claim 3, wherein the inorganic solvent comprises an inorganic compound comprising a carbonate, a pyrophosphate, or a tetraborate; or mixtures thereof.

10. The method of claim 3, wherein the inorganic solvent comprises an inorganic compound selected from K₄P₂O₇ or Na₄P₂O₇, or mixtures thereof.

11. The method of claim 3, wherein the organic solvent is selected from one or more of ethanol, methanol, 1-propanol, 2-propanol, dioxane, dimethyl sulfoxide, tetrahydrofuran, and dimethylformamide.

12. The method of claim 11, wherein the organic solvent is ethanol.

13. The method of claim 1, wherein step (a) produces a mixture in which the coal to liquid ratio is from 1 wt % to 5 wt %.

14. The method of claim 13, wherein step (a) produces a mixture in which the concentration of inorganic compound is 0.005M to 0.5M.

15. The method of claim 13, wherein step (a) produces a mixture in which the volume of the organic solvent is from about 3 vol % to about 40 vol %.

16. The method of claim 1, wherein the liquid phase from step (a) is separated from the coal sample prior to step (b).

17. The method of claim 1, wherein step (b) uses UV/VIS spectrophotometry to determine the concentration of extracted species in the liquid phase.

18. The method of claim 1, wherein the analysis of step (b) provides a value of the degree of surface oxidation of the coal.

19. A method for controlling collector used in a coal flotation process, the method comprising determining a degree of oxidation of coal by the method of claim 1 and controlling a ratio of non-polar collector to polar collector in the coal flotation process in response to the determined degree of oxidation of coal.

20. A method for controlling collector used in a coal flotation process, the method comprising determining a degree of surface oxidation of coal by the method of claim 18 and controlling a ratio of non-polar collector to polar collector in the coal flotation process in response to the determined degree of surface oxidation of the coal.
